(19) United States
(12) Patent Application Publication
(10) Pub. No.: US 2008/0262320 A1
(43) Pub. Date: Oct. 23, 2008

(54) System for Monitoring a Physical Parameter of a Subject

(76) Inventors: Timothy M. Schaefer, Rochester, MN (US); Daivd R. Holmes, Rochester, MN (US); Jonathan D. Coker, Rochester, MN (US); Edward B. Welch, Nashville, TN (US); Barry K. Gilbert, Rochester, MN (US); Kenton R. Kaufman, Rochester, MN (US)

Correspondence Address: QUARLES & BRADY LLP, 411 E. WISCONSIN AVENUE, SUITE 2040, MILWAUKEE, WI, 53202-4497, US

(21) Appl. No.: 11/993,735

(22) PCT Filed: Jun. 28, 2006
(86) PCT No.: PCT/US06/25034

§ 371 Date: Mar. 19, 2008

Related U.S. Application Data
(60) Provisional application No. 60/694,709, filed on Jun. 28, 2005.

(51) Int. Cl. A61B 5/00

(52) U.S. Cl. 600/300

(57) ABSTRACT

A system for monitoring a physical parameter of a subject includes a sensor, a transmitter, a repeater, and a base station. The sensor is located within the body of the subject and is operable to sense a physical parameter of the subject. The transmitter is electrically connected to the sensor, and is operable to transmit a spread spectrum encoded signal using a digital spreading code, the encoded signal carrying information indicative of the sensed physical parameter and being transmitted to a local region outside the body. The repeater is located in the local region, is movable with the subject, and is operable to receive the transmitted encoded signal and retransmit it to a remote region outside the body. The base station is located in the remote region and is operable to receive and decode the retransmitted encoded signal.

### CROSS-REFERENCE TO RELATED APPLICATIONS

This application is based on provisional application Ser. No. 60/694,709 filed Jun. 28, 2005 and entitled “Remote System for Monitoring Core Temperature” and claims the benefit thereof.

### STATEMENT REGARDING FEDERALLY SPONSORED RESEARCH OR DEVELOPMENT

Not Applicable

### FIELD OF THE INVENTION

The invention relates to a system for monitoring a physical parameter such as the core body temperature of a subject, especially in situations where individuals may experience hypothermia or hyperthermia as a result of heavy exercise, environmental conditions, or disease.

### BACKGROUND OF THE INVENTION

Warm blooded animals, such as humans, maintain a remarkably constant body temperature despite large variations in environmental temperatures. The internal temperature of the human body is maintained around 37 degrees centigrade (C) and typically moves up and down very little over the course of a day. This homeostasis of body temperature is of utmost importance because healthy survival depends on biochemical reactions taking place at certain rates. These rates, in turn, depend on normal enzyme functioning which depends on body temperature staying within the narrow range of normal. This enables humans to live under the extremes of temperature ranging from the very cold to the very hot. Homeostasis is essential for the maintenance of health and its breakdown results in serious consequences.

The environment in which competitive athletes or military personnel undergo physical conditioning may include extremes of hot and cold and these individuals may be pushed to their physiological limits. As documented by researchers in the field of thermoregulation, the human body is ineffective in hot environments such as encountered when running in hot conditions, at elevated core body temperatures, or in very cold environments such as encountered during lengthy cold-water swims. As a result, the body enters hyperthermic or hypothermic conditions and human function begins to deteriorate. Research highlights detrimental reactions of the body to hypothermia or hyperthermia. These reactions range from loss of dexterity to unconsciousness and may even lead to death. In the past several years, several professional athletes have died as a result of hyperthermia during practice sessions. To protect professional and amateur athletes undergoing rigorous training, a considerable safety measure can be gained through the use of a minimally-invasive device to monitor the core body temperature of these individuals during training.

Several methods are commonly used for monitoring core body temperature. If exact core temperature is needed, the pulmonary artery is the site of choice and is considered the “gold standard” because the observed temperature is a result of the convective mixing of blood from all over the body. Other authors consider the tympanic membrane temperature to best represent the core body temperature. A study of 20 elderly post-operative patients comparing pulmonary artery blood temperature to tympanic membrane temperature found no significant difference between the two sites. However, measurement from either of these sites is invasive and potentially dangerous. Further, environmental conditions such as encountered when swimming or exercising may prevent accurate measurement from these sites.

The sites most commonly used in clinical practice to measure core temperature are the axilla (armpit), mouth, and rectum. Although the axilla is safe and easily accessible, it is considered to be less accurate and more easily influenced by the environment and other variables. The mouth is often used as a site for temperature reporting since the sublingual pockets respond very quickly to changes in core body temperature. However, the oral temperature is not the actual core body temperature. One study found that the oral temperature was 0.37 degrees C. above pulmonary artery blood temperature. Another study compared oral and tympanic membrane temperatures in 60 patients and found that body temperature was 0.6 to 0.8 degrees C. higher at the tympanic site than the oral site in 99 percent of the measurements. Rectal temperature has traditionally been considered more accurate than either oral or axillary readings. This is due to the fact that the rectum has a good arterial blood supply via the hemorrhoidal artery, is well insulated, and is thought to be less influenced by external factors. However, rectal readings are consistently higher than core body temperature. Research has shown that the mean difference between rectal and pulmonary artery blood temperature is 0.26 degrees C. Moreover, the rectal temperature response to changes in the core body temperature is not as fast as the oral site. Other disadvantages to the use of the rectal site may include patient discomfort, pain, and embarrassment. Hazards may include mucosal injury, infection, and cross infection. These studies clearly demonstrate that the accuracy of a temperature measurement is strongly influenced by the choice of measurement site and the sites most commonly used in clinical practice do not accurately reflect core temperature.

Thus, an important consideration when comparing sites for body temperature measurements is the ability of each to accurately approximate core temperature (pulmonary artery blood). Ideally, the temperature difference should not exceed 0.2 degrees C., which is generally considered clinically significant. The utility of a temperature monitoring system is dependent on the accurate identification of core temperatures that exceed known physiological upper and lower thresholds.

Recently, an ingestible capsule for measuring temperature and wirelessly transmitting the measured temperature to an external receiver has been developed by the Johns Hopkins University Applied Physics Laboratory in collaboration with NASA's Guided Space Flight Center. This core body temperature monitoring system has been marketed under the trade name CorTemp™ by HQ Inc. of Palmetto, Fla. The ingestible capsule in this system includes a transmitter, a microbattery, and a quartz crystalline temperature sensor. Once inside the gastrointestinal tract, the crystal sensor vibrates at a frequency relative to the temperature of its surroundings, i.e., the body, producing a magnetic flux signal which is then wirelessly transmitted to the external receiver, which is part of an ambulatory data recorder. The data recorder picks up, displays, and stores the data in a solid state memory until the data is downloaded into a personal computer. However, the physical range of use of the data recorder is limited, due to the low power constraints on the capsule.

Thus, while such a wireless monitoring system is valuable because it minimizes the invasive nature of the temperature measurement, it is also desirable to that such a system be operable to monitor one or more individuals over a greater distance than has previously been possible.

### SUMMARY OF THE INVENTION

In one aspect, the invention is a system for monitoring a physical parameter of a subject including a sensor, a transmitter, a repeater, and a base station. The sensor is associated with the body of the subject and is operable to sense a physical parameter of the subject. The transmitter is electrically connected to the sensor and is operable to transmit a spread spectrum encoded signal using a digital spreading code, the encoded signal carrying information indicative of the sensed physical parameter and being transmitted to a local region outside the body. The repeater is located in the local region and is operable to receive the transmitted encoded signal and retransmit it to a remote region outside the body. The base station is located in the remote region and is operable to receive and decode the retransmitted encoded signal, and remotely monitor the physical parameter of the subject. Digital spread spectrum communication is robust, allows for asynchronous communication, and allows for the transmitted signal to travel longer distances with relatively low power.

In another aspect, the invention is a system for monitoring a physical parameter of a subject including a sensor, a transmitter, a repeater, and a base station. The sensor is located within the body of the subject and is operable to sense a physical parameter of the subject. The transmitter is electrically connected to the sensor and is operable to transmit a signal to a local region outside the body. The transmitted signal includes information indicative of the sensed physical parameter. A repeater is located in the local region, is movable with the subject, and is operable to receive the transmitted signal and retransmit it to a remote region outside the body. The base station is located in the remote region and operable to receive the retransmitted signal and remotely monitor the physical parameter of the subject. Such a system is advantageous to monitor a physical parameter of a moving or physically active subject.

In another aspect, the invention provides a system for monitoring the core body temperature of a subject, and includes a swallowable capsule, a temperature sensor, a transmitter, a repeater, and a base station. The temperature sensor is located inside the capsule and is operable to sense the temperature of the environment in which the capsule is immersed. The transmitter is located inside the capsule, is electrically connected to the temperature sensor, and is operable to transmit a radio frequency signal which is indicative of the sensed temperature. The transmitted radio frequency signal is transmitted to a local region outside the body. The repeater is attached to the subject and is operable to receive the radio frequency signal and retransmit it to a remote region outside the body. The base station is located in the remote region and is operable to receive the retransmitted radio frequency signal and monitor the temperature of the capsule environment. The system provides remote monitoring of the individual core body temperature at distances exceeding one kilometer or farther if necessary. In this manner, a team physician or the like will be able to determine if the monitored individual is approaching either a hypothermic or a hyperthermic state, locate and treat the individual until such time that the core temperature returns to an acceptable level. Once the core body temperature has returned to an acceptable level, the individual is able to resume the physical activity.

The present invention provides a system for monitoring the core body temperature of a subject which is advantageous in terms of its accuracy, non-invasive nature, ability to operate in extreme environmental conditions, and high power efficiency. Further, using identification (ID) codes associated with each transmitter, the system can be used to monitor a plurality of subjects over a fairly wide geographic range, e.g., a circle having a radius of at least a kilometer.

These particular objects and advantages may apply to only some embodiments falling within the claims and thus do not define the scope of the invention.

### BRIEF DESCRIPTION OF THE DRAWINGS

FIG. 1 illustrates in block diagram form a system for monitoring a physical parameter of a subject;

FIG. 2 illustrates an implementation of the transmitter and repeater of the system of FIG. 1;

FIG. 3 illustrates a slightly different implementation of the transmitter and repeater;

FIG. 4(a) illustrates the acquired data from four simultaneously transmitting transmitters and FIG. 4(b) illustrates the decoded data, showing a proper identification of the individual transponders by individual ID codes.

### DETAILED DESCRIPTION OF THE PREFERRED EMBODIMENT

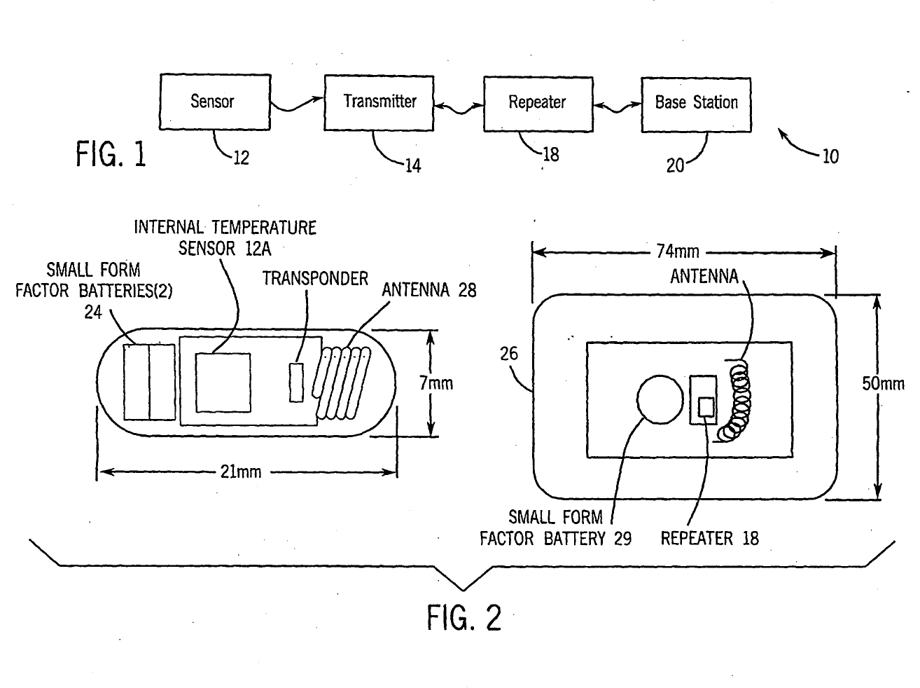

A system 10 for monitoring a physical parameter of a subject is illustrated in FIG. 1. System 10 includes at least one sensor 12 associated with the body of the subject for sensing a physical parameter of the subject, a transmitter 14 for transmitting a signal including information indicative of the sensed physical parameter, a repeater 18 for receiving and retransmitting the transmitted signal, and a base station 20 for receiving the retransmitted signal.

The sensor 12 can be a temperature sensor for measuring core body temperature, a heart rate sensor for measuring heart rate, an accelerometer for measuring acceleration of the subject in one or more directions, a pressure sensor for measuring a blood pressure, a pH sensor, a pO₂ sensor, or an imaging sensor, such as a small solid state CCD camera, or any other type of sensor for measuring a physical parameter of a subject. Although not specifically illustrated in FIG. 1, an A/D converter may be necessary to convert an analog signal from the sensor to a digital signal representative of the sensed physical parameter which is provided to the transmitter 14.

Transmitter 14 can include an activation circuit (not shown) that is controlled by a remote actuation signal to power the transmitter 14 on and off. In this manner, power to the transmitter 14 can be conserved as desired. The activation signal for the transmitter 14 can be provided by either the repeater 18 or the base station 20.

In a preferred embodiment, the transmitter 14 takes the form of a transponder that transmits a spread spectrum encoded signal which carries information indicative of the sensed physical parameter in response to an interrogation signal. The encoded signal is generated using a spreading code such as generated by a code generator component of the transponder. The transponder transmits the encoded signal in response to the interrogation signal to a local region outside the body. The transponder can use the interrogation signal to generate a transmit clock signal that is the same as the frequency of the interrogation signal or an integral division thereof. This eliminates the need for a separate clock component in the transponder.

The implementation of the above-described transponder form of transmitter 14 and its associated encoding circuits are described in pending patent application Ser. No. 10/915,576, published as US 2006/0034348, and titled “Asynchronous Communication System for Remote Monitoring of Objects or an Environment”. This application is hereby incorporated by reference. Additionally, the decoding or detector circuit for decoding the transmitted digital coded signal is described therein.

Preferably, the repeater 18 receives and retransmits the encoded signal and the base station 20 receives and decodes the retransmitted encoded signal to obtain the sensed physical parameter information. The transmitter 14, repeater 18, and base station 20 communicate wirelessly using radio frequency signals and in an asynchronous manner.

The encoded signal may additionally carry information from more than a single sensor and/or information indicative of an ID code specific to the transmitter 14. With the use of one or more sensors 12 and ID codes, the base station 20 can monitor one or more physical parameters of each of a plurality of subjects. For example, in the preferred embodiment, each transmitter 14 may have a specific ID code hard-wired, programmed or otherwise associated with that transmitter such that the ID code is encoded along with the sensed physical parameter information. The decoded ID code of the transmitter will identify the source of the associated sensed physical parameter information. A code generator component can use the ID code and sensed physical parameter information as seed data to generate the spreading code.

The use of spread spectrum communication between the transmitter 14, repeater 18, and base station 20 allows multiple transmitters to transmit simultaneously using the same transmit frequency with reduced interference. Also, the transmitter 14 may send data intermittently or in a pulsed manner. Furthermore, use of the asynchronous code division multiple access (CMDA) techniques may reduce the size, complexity, and power requirements of the transmitter 14.

In a preferred embodiment, the transmitter 14 uses digital spread spectrum signal processing to transmit data as “packets” or “frames” using so-called “orthogonal codes” or “quasi-orthogonal” codes such as binary Gold codes. Each packet is a digital stream of bits that contains ID information specific and unique to the particular transmitter 14 as well as a data packet including information representative of the sensed physical parameter. The base station 20 receives and decodes the signal transmitted by the repeater 18, using the same Gold codes and correlation to decode the signal. The base station 20 is able to separate the information transmitted from multiple transmitters.

The use of the orthogonal codes gives the base station 20 a detection advantage in that the unique ID codes allow for processing gain which aids in the distant detection of these low-level signals. The robustness and low power required of the system 10 is thus advantageous to transmit over longer distances.

The repeater 18 is located in the local region outside the body of the subject, preferably such that it is movable with the subject. For example, the monitored subject may be an individual engaged in an activity such as running or swimming and it is advantageous to have the repeater move as the subject moves.

In one embodiment, the repeater 18 is operable to generate the interrogation signal for the transponder, perhaps in response to a request from the base station 20. In another embodiment, the base station 20 generates the interrogation signal for the transponder. In this manner, rather than have the transponder continuously transmitting, the transponder will transmit data only when required and can thereby conserve power.

As mentioned, the repeater 18 is operable to receive the transmitted signal, amplify it, and retransmit it to a remotely located base station 20. In a preferred embodiment, both the transmitter 14 and repeater 18 include ultra-compact, high efficiency RF transmitters and depending on the transmitted frequency can provide a signal at a distance of one kilometer or more.

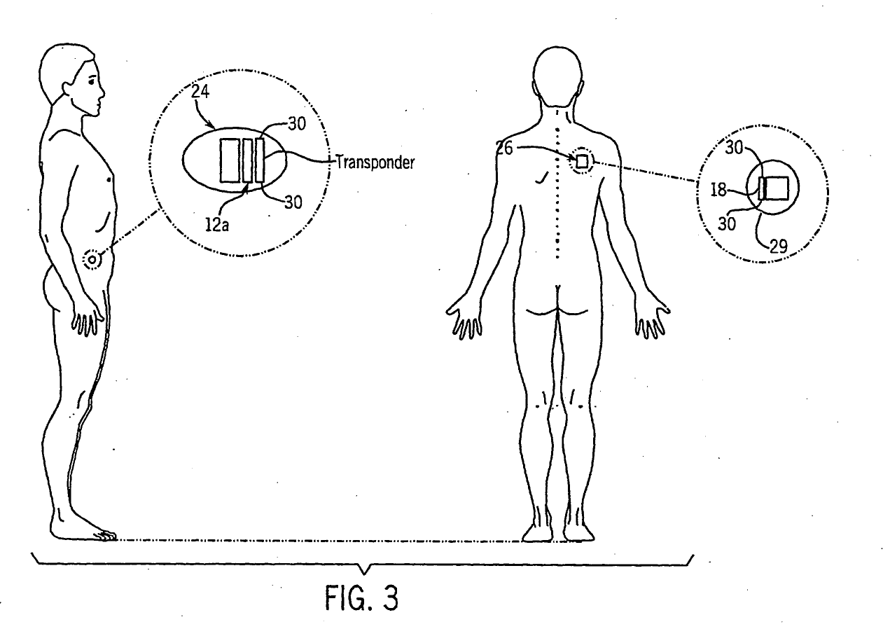

In implementations of the system such as is shown in FIGS. 2 and 3, the sensor 12 is a temperature sensor 12A which is placed along with transmitter 14 within an ingestible capsule 22 that can be swallowed by the subject to be monitored. The capsule 22 is preferably the size of a multivitamin so as to be easily swallowable, having for instance dimensions approximately 7 mm by 21 mm such as illustrated in FIG. 2 or 6 mm by 12 mm such as illustrated in FIG. 3. One or more small form factor batteries 24 are also provided to power the temperature sensor 12A and the transponder form of transmitter 14. Separate transmit and receive antennas can be provided in the capsule 22 such as shown in FIG. 3 or a single antenna 28 can be provided in the capsule such as shown in FIG. 2. As mentioned in paragraphs 38-40 of US Patent Application No. 2006/0034348, the physical size of a transponder and antenna can be made to be on the order of several millimeters or smaller.

Once swallowed by a subject, the capsule 22 remains for a period in the GI tract. The temperature sensor 12A can be a separate thermistor type sensor or can be a temperature sensitive solid state element incorporated directly on the same chip as the transmitter 14. The temperature signal from the sensor may be an analog signal which can be converted to a digital signal by an A/D converter. The digital signal from the A/D converter is used by the transmitter 14 and this information indicative of core body temperature of the subject is incorporated in the encoded data transmitted by the transmitter 14.

The repeater 18 can be connected to or within a patch 26 that can be directly attached to the body, such as with adhesive tape or the like, or indirectly attached to the body by being attached to clothing of the subject. The repeater 18 is thus movable with the subject. The repeater 18 includes at least one antenna 30, and is powered by a battery 29. Having the repeater 18 movable with the subject allows the accurate monitoring of the core body temperature of a subject, such as a runner, or a swimmer, who may travel relatively large distances. In the case of a subject in water, it may be advantageous to attach the repeater 18 to an area of the swimmer that is at least sometimes out of the water. For example, the patch 26 could be attached to the head of a subject.

The use of the repeater 18 is advantageous when the transmitter 14 is within the body or is located somewhere on the body but underwater. A signal transmitted through body tissue or through water will have a shorter range than a comparable signal transmitted through air. However, the swallowable capsule could also be used to monitor body core temperature or other physical parameters without the use of the repeater, by transmitting directly to the base station 20.

The base station 20 can also provide further analysis of the information transmitted. For example, the base station 20 can analyze the rate of change of the sensed temperature or other sensed parameter information, whether or not the measured core temperature of a subject or other sensed parameter information exceeds pre-defined upper and lower limits or is within a pre-defined acceptable range.

In this manner, the core temperature of the subject can be measured in an accurate manner and the sensed temperature information transmitted by the transmitter 14 to the repeater 18 and ultimately to the base station 20. The use of digital spread spectrum modulation of information is advantageous in order to enable the transmission of sensor information over longer distances than has previously been possible.

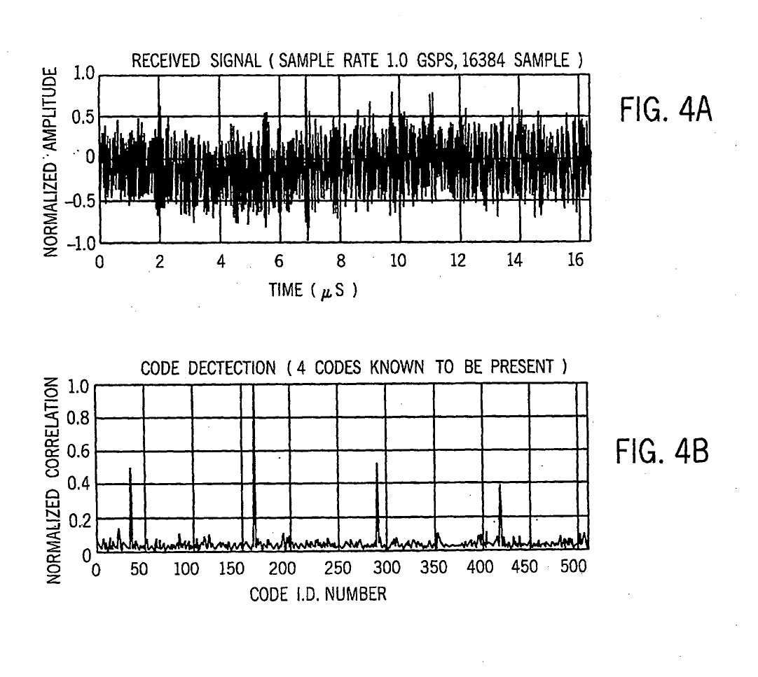

FIGS. 4(a) and 4(b) illustrate the acquired data from four simultaneously transmitting transmitters and the decoded data, showing a proper identification by individual ID codes of the four transmitters. In particular, the received signal shown in FIG. 4(a) was sampled at a rate of 1.0 giga-sample per second (GSPS) over an approximately 16 microsecond time frame. This example uses a 9-bit data packet and 3-bit address (ID code) to produce a total of 512 possible spreading codes. The graph shown in FIG. 4(b) illustrates the decoded data. The four peaks in the graph of FIG. 4(b) illustrate that the decoding process allows for the proper identification of signals from the four separate transmitters.

It is specifically intended that the present invention not be limited to the embodiments and illustrations contained herein, but include modified forms of those embodiments including portions of the embodiments and combinations of elements of different embodiments as come within the scope of the following claims.

We claim:

1. A system for monitoring a physical parameter of a subject, the system comprising:
a sensor associated with the body of the subject and operable to sense a physical parameter of the subject;
a transmitter electrically connected to the sensor and operable to transmit a spread spectrum encoded signal using a digital spreading code, the encoded signal carrying information indicative of the sensed physical parameter and being transmitted to a local region outside the body;
a repeater located in the local region and operable to receive the transmitted encoded signal and retransmit it to a remote region outside the body; and
a base station located in the remote region and operable to receive and decode the retransmitted encoded signal, and remotely monitor the physical parameter of the subject.

2. The system of claim 1, wherein the sensor is one selected from the group including a temperature sensor, a heart rate sensor, an accelerometer, a pressure sensor, a pH sensor, a pO₂ sensor, and an imaging sensor.

3. The system of claim 1, wherein the transmitter is a radio frequency transmitter.

4. The system of claim 1, wherein the transmitter is a transponder that transmits the encoded signal in response to an interrogation signal.

5. The system of claim 4, wherein the repeater generates the interrogation signal for the transponder.

6. The system of claim 5, wherein the repeater generates the interrogation signal for the transponder upon a request from the base station.

7. The system of claim 4, wherein the base station generates the interrogation signal for the transponder.

8. The system of claim 1, wherein the base station is operable to monitor a physical parameter of each of a plurality of subjects wherein each subject is associated with an individual transmitter having a corresponding identification code.

9. The system of claim 1, wherein the sensor and the transmitter are located within a swallowable capsule.

10. The system of claim 1, wherein power to the transmitter can be controlled by an activation signal provided by one of the repeater and the base station.

11. A system for monitoring a physical parameter of a subject, the system comprising:
a sensor located within the body of the subject and operable to sense a physical parameter of the subject;
a transmitter electrically connected to the sensor and operable to transmit a signal to a local region outside the body, the transmitted signal including information indicative of the sensed physical parameter;
a repeater located in the local region, movable with the subject, and operable to receive the transmitted signal and retransmit it to a remote region outside the body; and
a base station located in the remote region and operable to receive the retransmitted signal, and remotely monitor the physical parameter of the subject.

12. The system of claim 11, wherein the sensor is one selected from the group including a temperature sensor, a heart rate sensor, an accelerometer, a pressure sensor, a pH sensor, a pO₂ sensor, and an imaging sensor.

13. The system of claim 1, wherein the transmitter is a transponder that transmits the encoded signal in response to an interrogation signal.

14. The system of claim 13, wherein the repeater generates the interrogation signal for the transponder.

15. The system of claim 14, wherein the repeater generates the interrogation signal for the transponder upon a request from the base station.

16. The system of claim 13, wherein the base station generates the interrogation signal for the transponder.

17. The system of claim 11, wherein the base station is operable to monitor a physical parameter of each of a plurality of subjects wherein each subject is associated with an individual transmitter having a corresponding identification code.

18. The system of claim 11, wherein the sensor and the transmitter are located within a swallowable capsule.

19. The system of claim 11, wherein the transmitted signal is a spread spectrum encoded signal generated using a digital spreading code.

20. The system of claim 11, wherein power to the transmitter can be controlled by an activation signal provided by one of the repeater and the base station.

21. A system for monitoring core body temperature of a subject, the system comprising:
a swallowable capsule;
a temperature sensor inside the capsule and operable to sense the temperature of the environment in which the capsule is immersed;
a transmitter inside the capsule, electrically connected to the temperature sensor, and operable to transmit a radio frequency signal which is indicative of the sensed temperature, the transmitted radio frequency signal being transmitted to a local region outside the body;
a repeater attached to the subject and operable to receive the radio frequency signal and retransmit it to a remote region outside the body; and
a base station located in the remote region and operable to receive the retransmitted radio frequency signal and monitor the temperature of the capsule environment.

22. The system of claim 21, further including an antenna within the capsule.

23. The system of claim 21, further including at least one battery inside the capsule.

24. The system of claim 21, wherein the transmitter is a transponder that transmits the encoded signal in response to an interrogation signal.

25. The system of claim 24, wherein the repeater generates the interrogation signal for the transponder.

26. The system of claim 21, wherein an antenna is associated with the repeater.

27. The system of claim 21, wherein the repeater is on a patch that is attached to the body.

28. The system of claim 21, further including at least one battery to power the repeater.

29. The system of claim 21, wherein the transmitted radio frequency signal is a spread spectrum encoded signal generated using a digital spreading code.

30. The system of claim 21, wherein the base station is operable to monitor a physical parameter of each of a plurality of subjects wherein each subject is associated with an individual transmitter having a corresponding identification code.

31. A system for monitoring core body temperature of a subject, the system comprising:
a swallowable capsule;
a temperature sensor inside the capsule and operable to sense the temperature of the environment in which the capsule is immersed;
a radio frequency transponder mounted inside the capsule and electrically connected to the temperature sensor, the radio frequency transponder operable in response to an interrogation signal to transmit a radio frequency spread spectrum encoded signal using a digital spreading code, the transmitted encoded signal carrying information indicative of the sensed physical parameter and being transmitted to a local region outside the body;
a radio frequency repeater attached to the subject and operable to receive the transmitted encoded signal and retransmit it to a remote region outside the capsule, the repeater providing the activation signal for the transponder; and
a base station located in the remote region and operable to receive the retransmitted encoded signal and monitor the temperature of the capsule environment.

32. The system of claim 31, wherein the repeater is on a patch that is attached to the body.

33. The system of claim 31, wherein the base station is operable to monitor a physical parameter of each of a plurality of subjects wherein each subject is associated with an individual transponder having a corresponding identification code.
